(19) United States
(12) Patent Application Publication
(10) Pub. No.: US 2007/0219537 A1
(43) Pub. Date: Sep. 20, 2007

(54) Pressure gauge display for chest drainage unit

(76) Inventors: Trinh D. Phung, Attleboro, MA (US); Erich A. Dreyer, Bellingham, MA (US); Douglas W. Moore, Westwood, MA (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 11/501,757

(22) Filed: Aug. 10, 2006

Related U.S. Application Data
(63) Continuation-in-part of application No. 11/391,318, filed on Mar. 29, 2006.
Application No. 11/391,318 is a continuation-in-part of application No. 11/377,549, filed on Mar. 17, 2006.

(51) Int. Cl. A61M 27/00

(52) U.S. Cl. 604/541

(57) ABSTRACT

A pressure indicator for a chest drainage device includes an outer casing with a longitudinal axis and first and second end portions defining first and second openings. A flexible element is disposed inside the outer casing to define a collapsible pressure-holding inner space in fluid communication with the second opening. An indicator element is disposed inside the outer casing configured to translate along the longitudinal axis. The flexible element, indicator element and outer casing together define a variable interior space inside the outer casing in fluid communication with the first opening. A force resistance element is placed in the casing to resist the translation of the indicator element along at least one direction along the longitudinal axis when a pressure differential exists between the first and second openings. The pressure indicator is placed inside the chest drainage device to be viewable from multiple angles.

### CROSS-REFERENCE TO RELATED APPLICATIONS

The present application claims priority to and is a continuation-in-part of U.S. application Ser. No. 11/391,318, filed Mar. 29, 2006, now pending, which is a continuation-in-part of U.S. application Ser. No. 11/377,549, filed Mar. 17, 2006, now pending, the entire contents of which are incorporated herein by reference.

### FIELD OF THE INVENTION

The present invention relates generally to medical devices. More particularly, the present invention relates to a display means for a pressure indicating means in a chest drainage unit.

### BACKGROUND OF THE INVENTION

Chest drainage devices and systems and more particularly suction drainage systems and devices for removing gases and/or liquids from medical patients, such as from the pleural cavity, by means of a pressure differential, are well known in the art. For many years, the standard apparatus for performing the evacuation of the pleural cavity was a drainage system known as the “3-bottle set-up” which includes a collection bottle or chamber, a water seal bottle, and a suction control bottle. A catheter runs from the patient's pleural cavity to the collection bottle, and the suction bottle is connected by a tube to a suction source. The three bottles are connected in series by various tubes to apply suction to the pleural cavity to withdraw fluid and air and thereafter discharge the same into the collection bottle. Gases entering the collection bottle bubble through water in the water seal bottle. The water in the water seal also can prevent the back flow of air into the chest cavity. Suction or “negative” pressure is usually provided by a central vacuum supply in a hospital so as to permit withdrawal of fluids such as blood, water and gas from a patient's pleural cavity by establishing a pressure differential between the suction source and the internal pressure in the patient.

The 3-bottle set-up lost favor with the introduction of an underwater seal drainage system first sold under the name “Pleur-evac”® in 1966 by Deknatel Inc. U.S. Pat. Nos. 3,363,626; 3,363,627; 3,559,647; 3,683,913; 3,782,497; 4,258,824; and U.S. Pat. No. Re. 29,877 are directed to various aspects of the Pleur-evac® system, which over the years has provided improvements that eliminated various shortcomings of the 3-bottle set-up. These improvements have included the elimination of variations in the 3-bottle set-up that existed between different manufacturers, hospitals and hospital laboratories. A principal feature of the Pleur-evac® system is the use of a single, unitary, pre-formed, self-contained unit that embodies the 3-bottle techniques. The desired values of suction can be established by the levels of water in a suction control chamber. These levels are filled according to specified values prior to the application of the system to the patient. Alternatively, dry suction elements can be used and a pressure regulator element can be equipped to regulate the suction and therefore pressure conditions inside the various chambers of the chest drainage unit. In particular, variable, adjustable pressure regulators can be coupled to the flow pathways inside the chest drainage unit to control the suction pressure present inside the collection chamber of the device, and hence the pleural cavity of the patient which is directly in communication with said collection chamber. This can be achieved by modulating or regulating the amount of pressure regulation flow that the pressure regulator draws from the ambient air to mix with the suction flow being drawn by the suction source.

However this pressure regulation function is independent of the actual reading of the regulated pressure inside the device. Current methods of indicating patient pressure are inaccurate by design. Most methods indicate only the pressure at the suction source connection or the amount of flow proximate thereto. The pressure at the suction source connection and that at the collection chamber is assumed to be correct. However that is not always the case. Pressure, head, or other gas dynamic losses in a complex set of flow control elements and valves found in chest drainage devices can lead to significant pressure variations throughout the device, such that the pressure at the suction source and pressure at the collection chamber can be very different. For proper operation of a chest drainage device during surgery, it is desirable to monitor the pressure easily and accurately and directly as close to the patient as possible. For a chest drainage assembly, this usually means at the first chamber coupled to the patient, namely, the collection chamber.

Another problem with chest drainage devices is that an assembly or apparatus can be placed in a myriad of positions in the surgical environment, such that it might need to be viewed from any number of angles by surgical and medical personnel. This can lead to the particular problem of not being able to clearly view the displays and functional elements of the chest drainage device and all of its indicators during operation. This applies equally to any pressure indicator, where, as is well known in the field, any measure of pressure indicated by the device is particularly important during a surgical procedure. For example, if the chest drainage apparatus is placed on the floor, as is oftentimes the case during a surgical procedure, a pressure indicator on the apparatus would be far below the line of sight of any medical personnel standing up right. Any display elements that might be shown on the front face of the device will not be easily viewable. This would lead to a degraded performance for the overall assembly, as medical or surgical personnel would strain or have to move to properly view any pressure indicator or read-out displayed by the chest drainage device.

It is desirable therefore, to have a pressure indicator means in a chest drainage assembly that can be easily installed, viewed and read by medical personnel. It is further desirable to have a pressure indicator means that can be viewed from a number of angles in the surgical environment.

### SUMMARY OF THE INVENTION

The foregoing needs are met, to a great extent, by the present invention, wherein in one aspect an apparatus is provided that in some embodiments includes a chest drainage unit that can accurately and effectively display and read the pressure indicative of the actual pressure in a patient.

In accordance with one embodiment of the present invention, a pressure indicator for a chest drainage device is provided, including a pressure indicator device, and a chest drainage assembly housing body enclosing the pressure indicator device, the housing body defining a plurality of windows through which the pressure indicator device can be viewed.

In accordance with another aspect of the present invention, a pressure indicator for a chest drainage device is provided, including a pressure indicator means, and a housing enclosing the pressure indicator means, the housing defining a plurality of viewing angles for viewing a pressure indicator element in said pressure indicator means.

In accordance with yet another aspect of the present invention, a chest drainage assembly is provided, having a pressure gauge including a pressure indicator element translating along a longitudinal axis. An assembly housing encloses the pressure gauge. The assembly housing defines a top face defining a display for viewing the pressure indicator element.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a perspective view illustrating the separate components of a modular chest drainage device prior to final assembly.

FIG. 2 is a top view of the assembly shown in FIG. 1.

FIG. 3 is a schematic front view of a modular chest drainage device similar to that shown in FIG. 1, shown as assembled without the face plate.

FIG. 4 is a perspective view illustrating a flow control module in the chest drainage assembly shown in FIGS. 1-3.

FIG. 5 illustrates a pressure indicator device according to an embodiment of the present invention.

FIG. 6A is a longitudinal view of a pressure indicator of the present invention taken along section 6-6 in FIG. 5, showing the indicator in fully expanded position.

FIG. 6B is another view of the indicator shown in FIG. 6A, showing the indicator in a contracted position.

FIG. 7 is a detail front view showing a pressure indicator of the present invention installed and positioned inside a modular chest drainage device similar to that shown in FIG. 1, shown as assembled without the face plate, according to an embodiment of the invention.

FIG. 8 is a sectional view of another embodiment of a pressure indicator of the present invention.

FIG. 9 is a front sectional view of another embodiment of a chest drainage assembly.

FIG. 10A is a top view of the chest drainage assembly shown in FIG. 9.

FIG. 10B is an enlarged view of the pressure gauge display in the top view of the chest drainage assembly shown in FIG. 9.

### DETAILED DESCRIPTION

The invention will now be described with reference to the drawing figures, in which like reference numerals refer to like parts throughout. An embodiment in accordance with the present invention provides a pressure indicator for a chest drainage unit. The indicator includes an outer casing having a longitudinal axis and a first end with an opening exposed to ambient air and a second end with an opening coupled to communicate with the collection chamber inside a chest drainage unit for reading patient pressure. A linear force resistance element in the form of a spring compressed inside a bellows is disposed inside the outer casing and aligned along the longitudinal axis. An indicator cap is disposed inside the outer casing and coupled to a tip portion of the bellows element. The interior of the bellows communicates with the collection chamber pressure such that the spring and bellows expands and contracts inside the casing to indicate the degree of suction pressure in the collection chamber. The indicator can be installed in a space inside the body of the chest drainage unit or can be attached thereto. The indicator can give a true reading of patient pressure inside the collection chamber of the chest drainage device, allowing for more effective and safer use of the device and assembly. The pressure indicator is enclosed by the chest drainage assembly housing body which defines a plurality of windows or viewing angle for viewing the pressure indicator both from the frontal face of the assembly as well as the top, upward facing surface of the assembly.

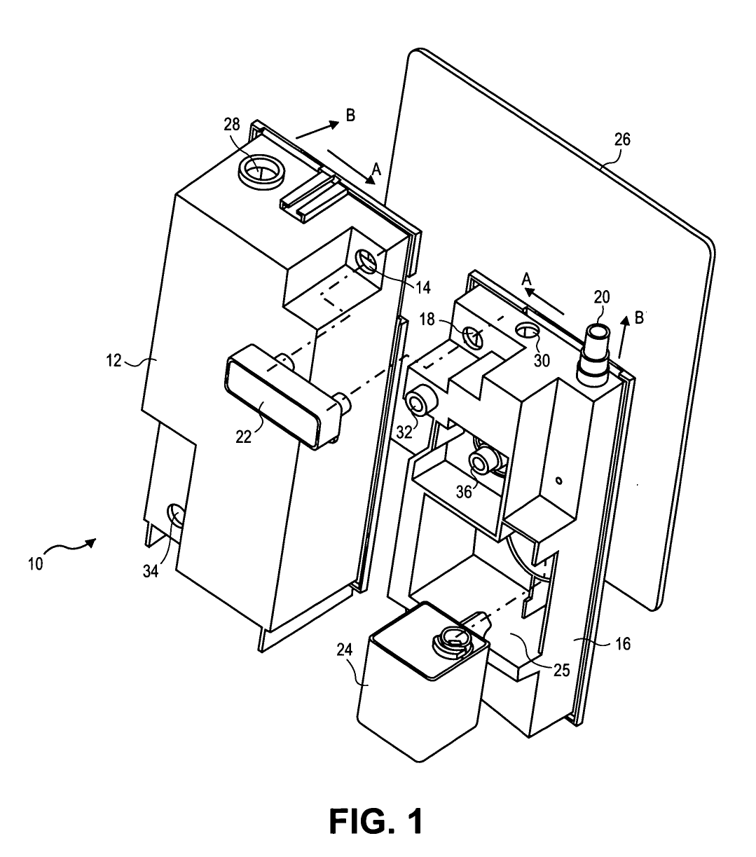

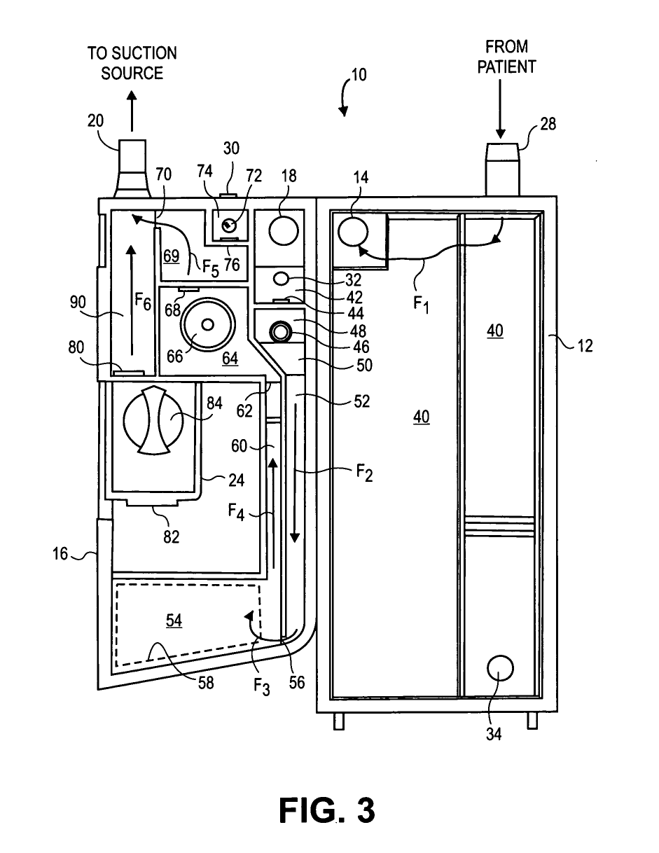

The pressure indicator device of the present invention can be fitted and installed in a chest drainage unit to read patient pressure as fluids are drained by the unit from a patient. A type of such a chest drainage unit is illustrated in FIG. 1. A modular chest drainage device 10 includes a collection module 12 defining a fluid collection chamber inside of it and having an exit port 14 for transmitting a suction flow out of the collection chamber. A ‘flow control’ module 16 defines an entry port 18 for receiving the suction flow from the collection chamber, a suction port 20 for coupling to a suction source (not shown), and a pressure regulation flow intake port (shown in FIGS. 3 and 4). A flow coupling 22 is provided between the exit port 14 and the entry port 18. A pressure regulation module 24 is sealingly coupled to the pressure regulation flow intake port on the flow control module 16 and can be positioned in an enclosure 25 defined by the walls and geometry of the flow control module 16 as shown in FIG. 1. The pressure regulation module 24 has an adjustable valve assembly therein for regulating a pressure regulation flow into the flow control module 16 from an ambient air intake port provided on the pressure regulation module 24. A face plate 26 is provided, wherein the collection module 12 and flow control module 16 are first aligned next to each other as in arrows A and then attached to face plate 26 as in arrows B so that the assembly can form multiple flow pathways, as will be illustrated in further detail below.

The collection module includes a fluid intake port 28 for receiving fluids from a patient. A catheter, tube, or similar device can be coupled to the fluid intake port 28 in a variety of ways as is well known in the art. An ambient air port 30 is included on the flow control module 16 as part of a positive pressure relief valve element therein. A filling valve 32, such as a grommet or needle-less fill valve with a luer type fitting, is provided on the flow control module 16 for injecting fluids into the module for filling a manometer chamber or water seal chamber that is needed to control the backflow of gases and to indicate pressure, flow, or breathing, as further explained below. A re-infusion port 34 is provided on the collection module 12 for allowing collected body fluids to be returned to a patient by a re-infusion line. A high negativity pressure relief valve 36 is also provided on the flow control module 16 to prevent excessive negative pressures from building in the device. A small room air entry port or opening 39 is also defined on the flow control module 16, for allowing communication with the pressure indicator assembly of the present invention as explained more fully below.

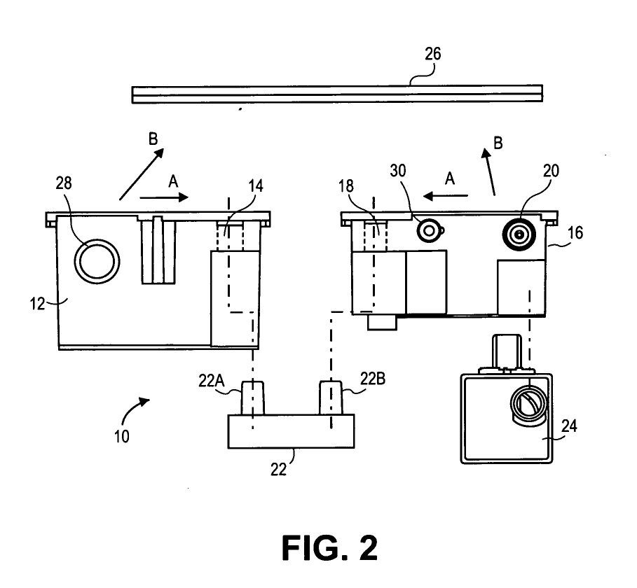

FIG. 2 is a top view of the assembly shown in FIG. 1. After the collection module 12 and flow control module 16 are aligned in the direction of arrows A to be positioned right next to one another, the flow coupling 22 is attached or coupled, permanently or detachably, to the collection module 12 through the flow exit port 14 with tubular extension 22a, and to the flow control module 16 through flow entry port 18 with tubular extension 22b. Thus, fluid or pressure communication, or a flow pathway, is established between the collection chamber inside of the collection module 12 and the flow pathways inside of flow control module 16.

FIG. 3 is a schematic front view of a modular chest drainage device 10 similar to that shown in FIG. 1, shown as assembled without the face plate. Fluid entering the device 10 from a patient first passes through the fluid intake port 28 and enters the collection chamber 40 defined inside the walls of the collection module 12. The collection chamber 40 can be made up of any number of compartments or sub-compartments, as is well known in the art, and can vary in size depending on the nature of the patient body to which the chest drainage device is attached: i.e. adult vs. pediatric sizes. Suction pressures established throughout the device 10 are also present in the collection chamber 40 such that gases entering the collection chamber 40 are passed out of the chamber through an exit port 14, while the liquid matter in the fluids captured inside of the collection chamber 40 remains trapped inside the chamber. Suction pressure is thereby ‘transmitted’ throughout the collection chamber via port 14, such that a ‘suction flow’ F1 is established between the intake port 28 and exit port 14. As used herein, the term ‘suction flow’ shall mean either a flow of gases or fluids from one point to another driven by a source of suction, or a flow in the direction of a negative pressure gradient, or an actual negative pressure gradient itself.

After exiting the collection chamber 40, the suction flow is transmitted though the flow coupling 22 and enters the flow control module 16 through entry port 18. The flow then proceeds downwards according to the orientation of view in FIG. 3, into sub-compartment 42 which is in communication with the fill valve 32. The flow then passes through a hole 44 having a valve-seat shaped on its underside, under which a ball element 46 is disposed in another sub-compartment 48. The flow passes though this sub-compartment 48 past a ramped funnel compartment 50 into an arm 52 which, when filled with fluid, serves as part of a water seal element, which can be filled with fluid injected from fill port 32. The flow then proceeds in the direction F2 though the water seal element, which can also function as a breathing indicator manometer.

Thus, the ‘suction flow’ can be transmitted along arrow F2 through the manometer in arm 52 into the water seal chamber 54 via flow arrow F3 which enters through a narrow opening 56 at the bottom of arm 52. An air leak indicator and metering element 58 can be included in chamber 54 as is well known in the art. Flow can then continue along pathway F4 through another passage or arm 60, past another opening 62, and into chamber 64. A high negativity pressure relief valve 66 can be disposed on the flow control module 16 to place chamber 64 in fluid communication with ambient air outside the device when the pressure inside said chamber 64 exceeds a pre-determined negative pressure (gauge or absolute, as the case may be). The flow proceeds though another opening 68 into chamber 69 and along arrow F5 past an opening 70 and into the suction port 20 for capture by the suction source. Thus, the ‘suction flow’ or suction pressure can be transmitted through the device 10, from intake port 28 to exit 20.

When the face plate 26 is bonded to the flow control module 16 and collection module 12, at least a first fluid flow passageway is defined from the entry port 18 on the flow control module 16, through sub-compartments 42 and 48, down through the arm 52, through chamber 54 and arm 60, into chamber 64, and up out though opening 70 into suction port 20, as shown generally along flow arrows F2, F3, F4, and F5. A positive pressure relief valve element is also included into the form of a ball 72 inside sub-compartment 74 above an opening 76.

The pressure regulator module 24 is shown to be sealingly coupled or attached to the flow control module 16 as shown in FIG. 3, through the pressure regulation flow intake port 80. The pressure regulator module 24 has an ambient air intake port 82 though which room air at non-suction pressures can be sucked though the pressure regulator 24. The pressure regulator 24 includes a user-adjustable dial element 84 which can be accessed through a hole fitted in the face plate (not shown). When the pressure regulator 24 is open, room air is allowed into the flow control module 16 to equalize pressures and flows along a second flow pathway in said module along arrow F6 as shown. This ‘pressure regulation flow’ mixes with the suction flow F5 just before the suction port 20 to control the operating pressures inside the device 10, such as in the collection chamber 40.

FIG. 4 is a perspective view illustrating a flow control module of the present invention in accordance with one embodiment. An access hole 92 is provided in a front panel 94 of the flow control module 16, through which the adjustable controls of the pressure regulation module 24 (not shown) would be accessible when the pressure regulation module is assembled with the flow control module 16. A horizontal shelf 96 is also shown in arm 60 having an opening 98 at the back end of shelf 96 away from the front panel end of the flow control module, and provides the means for flow F4 to enter through to opening 62 and on into chamber 64.

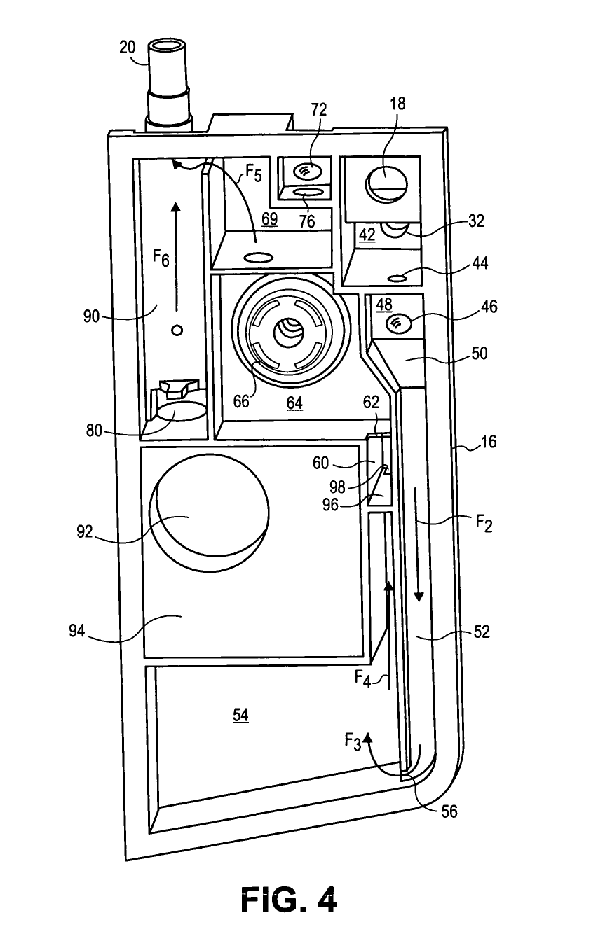

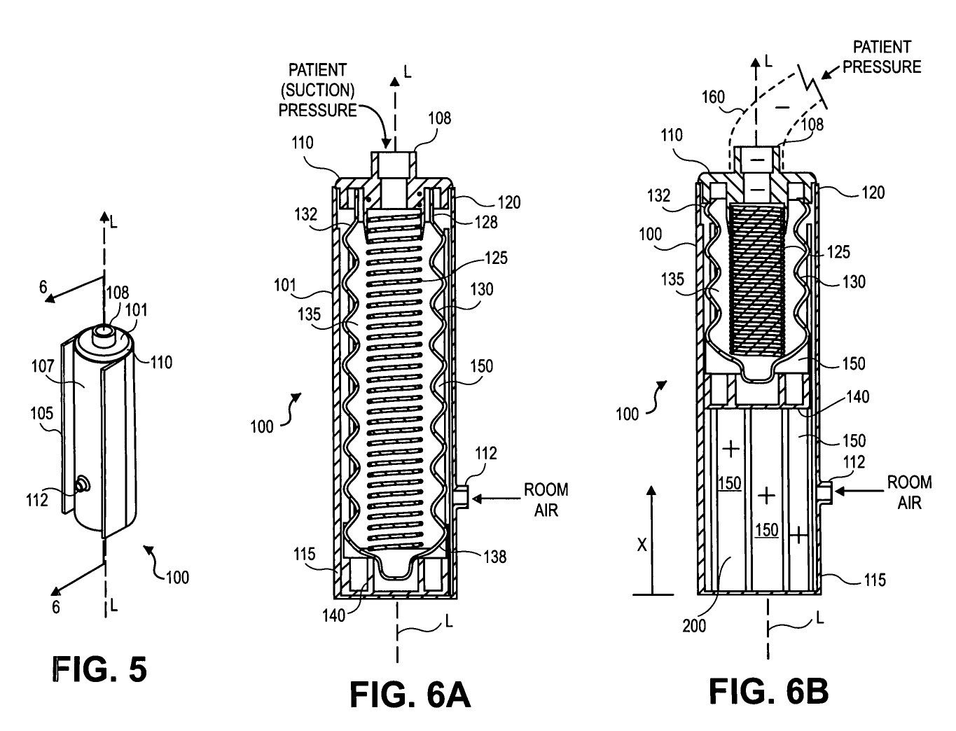

FIG. 5 illustrates a pressure indicator device according to an embodiment of the present invention. The patient pressure gauge or indicator assembly 100 includes a cylindrical body or outer casing 101 having a longitudinal axis “L”. Although body 101 can have a variety of other shapes, such as a rectangular prism, in the case of the cylindrical body shown in FIG. 5, the longitudinal axis L also defines an axis of symmetry around which the cylindrical body is centered. The indicator 100 also includes a wrap-around sheet-like surface element 105, such as a silkscreen, which can be printed with markers and gradations which is wrapped around a portion of the side surface 107 of the casing 101 as shown in FIG. 5. Alternatively, markings can be made directly onto the outer surface of the casing 101. The cylindrical casing 101 defines two openings, a central axial opening 108 located at one end portion or cap 110 of the body 101; and a lower end opening 112 defined in a portion of the side surface 107 nearer to the end portion of the body 101 opposite to the cap 110.

FIG. 6A is a longitudinal view of a pressure indicator of the present invention taken along section 6-6 in FIG. 5, showing the indicator in fully expanded position, without the wrap-around surface element 105. The pressure indicator 100 includes the outer body or casing 101 having a longitudinal axis L. The body 101 includes a first end portion 115 and a second end portion 120, where the first end portion 115 can be generally referred to as the ‘extension’ end portion of the indicator and the second end portion 120 can be referred to as the ‘base’ end of the indicator. The first end portion defines the first opening or port 112 on the casing 101. The second end portion defines the second opening or port 108, which is centrally oriented around axis L on the base end cap 110.

The indicator assembly 100 further includes a force resistance element 125 disposed inside outer casing 101 and aligned along the longitudinal axis L, the force resistance element 125 having a base end 128 attached to the second end portion 120, on an opposite side of the cap 110 as shown. As used herein, the term ‘force resistance element” shall mean any device, mechanism, or element which provides a means to resist an applied external force with a responsive counter-force. As used herein, a “linear force resistance element” shall mean any force resistance element whose responsive counter-force is a linear function of a displacement, translation or contraction of a portion of the linear force resistance element. An example of a linear force resistance element can be a spring. However the present invention encompasses and contemplates any type of force resistance element, such as those produced by a variety of mechanical, electrical, hydraulic, pneumatic, magnetic, or other means well known in the art. In the embodiment shown in FIG. 6A, force resistance element 125 is a spring.

A bellows element 130 is disposed inside the outer casing 101 around the force resistance element 125. The bellows element 130 include a base open end 132 attached proximate the second end portion 120 onto the base end 128 of cap 110 around the second opening 108. The bellows element 130 defines a collapsible inner space 135 in fluid communication with the second opening 128. The bellows element 130 includes a tip portion 138 opposite the base open end 132. A indicator cap 140 is disposed inside the outer casing 101 and coupled to the tip portion 138 of the bellows element 130. Due to the undulating surface of bellows element 130 positioned inside the casing 101, and a narrow annular tolerance space between the indicator cap 140 and the case 101 which allows the indicator cap 140 to slide up and down inside said casing, a variable interior space 150 is defined inside the outer casing 101, which is in communication with the first opening 112.

In operation, the indicator assembly is positioned to receive ambient room air through first opening 112, which fills the variable interior space 150. The second opening 108 is coupled to a pressure holding space, such as the collection chamber of a chest drainage unit that is under suction pressure. Thus, the negative suction pressures are communicated through the opening 108 into the collapsible inner space 135 defined by the bellows element 130 and around the spring 125. A lower pressure inside space 135 and a higher pressure inside space 150 creates a pressure differential that will cause the bellows element 130 and corresponding inner space 135 to collapse and contract. This pressure differential acts as an externally applied force against the force resistance element 125, which will resist the contraction of the bellows 130. As bellows 130 contracts in the direction of axis L, the spring element 125 will provide a counterforce in the opposite direction.

Thus the bellows element 130 and force resistance element 125 expand and contract along the longitudinal axis L inside the outer casing 101 in response to a pressure differential between the first opening 112 and second opening 108. FIG. 6B is another view of the indicator shown in FIG. 6A, showing the indicator assembly in a contracted position. A relatively higher pressure (indicated by the ‘+’ symbols in FIG. 6B) is present inside the variable interior space 150, which is exposed to room air through opening 112; and a relatively lower pressure (indicated by the ‘−’ symbols in FIG. 6B) is present inside the bellows 130 and its inner space 135 which is in direct fluid communication with the ‘patient pressure’ through a line, tube, catheter, or other connection means 160. As used herein, the term ‘patient pressure’ shall mean a pressure indicative of the pressure inside a patient's body, to which a chest drainage unit applying suction pressures is connected, and can mean the pressure inside a collection chamber defined by the chest drainage unit, which collection chamber is directly in fluid communication with the patient through a pressure holding direct fluid pathway or passageway.

If the force resistance element 125 is a linear element such as a spring, the counter-resistance of the spring will be proportional to the displacement ‘X’ of the indicator cap 140. As such, calibration of the pressure indicator assembly 100 can be carried out by coupling the device to a known pressure or pressure differential and using that as a ‘set point’ to mark the assembly. Such calibration can occur either within or outside of a chest drainage assembly. This provides a significant advantage in that if calibration is done prior to installation of the indicator component in a chest drainage assembly, the indicator can be easily replaced if the calibration shows structural or function problems with the device. The range of pressures can then be derived from the set point based on the resistive properties of the spring 125. The wrap-around sleeve 105 shown in FIG. 5 can thereby be a printed surface element wrapped around the exterior surface of the outer casing 101, said printed surface element having markings to indicate pressure, based on the relative displacement position X of the indicator cap 140. If the force resistance element 125 has a non-linear response, the pressure indicator assembly 100 can be calibrated based on the non-linear response curve and the appropriate markings made. Thus, the pressure indicator assembly 100 described herein can be operated to indicate patient pressure and can be practically viewed by a user for ease and accuracy via the movement of the bellows 130 and indicator cap 140.

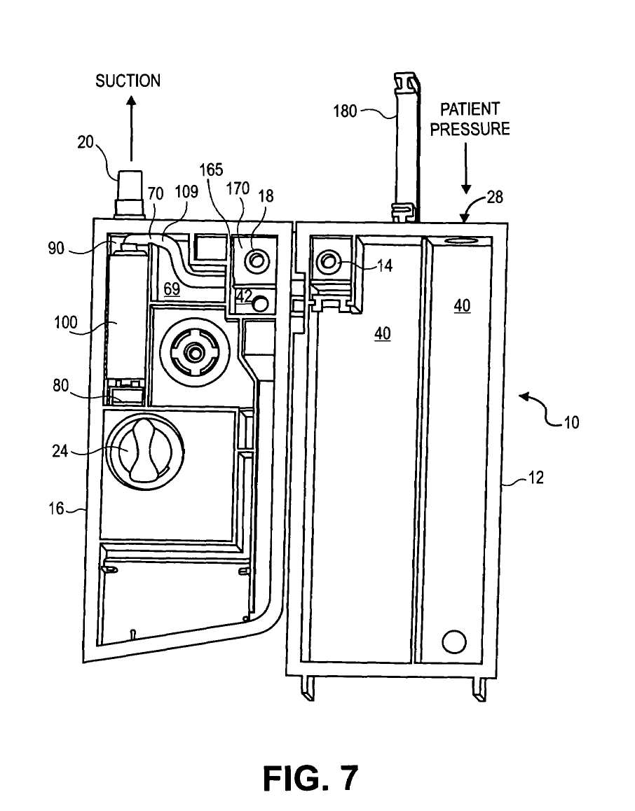

FIG. 7 is a detail front view showing a pressure indicator 100 of the present invention installed and positioned inside a modular chest drainage device 10 similar to that shown in FIG. 1, shown as assembled without the face plate, according to an embodiment of the invention. Suction is applied to the suction port 20, which, as explained above with reference to FIGS. 1-4, will be transmitted via the pathways formed inside flow control module 16 to the entry port 18, through the flow coupling 22 (not shown in FIG. 7), past the exit port 14, through to collection chamber 40. Collection chamber is directly coupled to a patient via intake port 28, and will therefore be closest to the pressure inside the patient. The pressure indicator assembly 100 is placed inside chamber 90 which is positioned between the suction port 20 and pressure regulation flow intake port 80. However the flow F6 between these two points is not blocked or ported into the pressure indicator 100, but rather flows around the indicator 100 because of the crevices and recesses formed between the indicator 100 and chamber 90, such as when the indicator body 101 is cylindrical. Thus the pressure indicator is not coupled to either the suction port 20 or the pressure regulator module 24. Instead, the opening 112 on the indicator 100 is positioned to communicate through opening 39 shown in FIGS. 1 and 4 as defined by the body of the flow control module 16, which is exposed to room air. The other opening 108 on the pressure indicator 100 is coupled to communication tubing 109, which, in the embodiment of FIG. 7 is shown to be routed through the inside of the flow control module 16, though opening 70, chamber 69, and through a divider wall 165 and into either the flow coupling 22 or a chamber 170 (directly above and communicating with the chamber 42) which is directly downstream in the suction flow path of the flow coupling 22 past entry port 18, to thus communicate with the collection chamber 40 directly through the flow coupling 22. The communication tubing 109 can also be routed in alternative ways, either inside or outside of the body of the flow control module 16 or collection module 12, as long as the tubing 109 is connected directly into a space that is in direct fluid communication with the patient, without any valves, water seals, measuring devices, indicators, manometers, and the like, which would deviate from the ‘true’ patient pressure.

Thus when the face plate 26 is applied to the assembly 10, it will have either a window, opening, or non-opaque element that will allow a user to view the movement of the indicator cap 140 in the pressure indicator assembly 100. Such a window or viewing element could also include markings to measure the degree of movement of the indicator cap 140, if said markings were not included on the outer casing 101 or silkscreen 105. In addition, a light-absorbing or glowing material could be applied to the elements of the indicator assembly 100, such as the indicator cap 140, or the inside surface 200 (shown in FIG. 6B) of the outer casing 101, to allow a viewer to easily ascertain the position of the indicator cap 140, and hence the pressure reading, in low light environments. This could be particularly useful in a hospital environment where quick readings at low light are often necessary.

Overall, the subject invention presents many advantages over the prior art when using a chest drainage device, such when dialing down pressure, where the pressure indicator 100 of the present invention allows real-time measurement of the change in pressure at the patient end, while known chest drainage devices can include a check valve element that can hold the pressure inside the flow pathways of the chest drainage unit, causing the prior art pressure indicators inside the chest drainage assembly to indicate a pressure different from that of the true patient pressure measurable by the present invention. Other advantages include: (i) being able to read the true patient pressure when the source suction pressure is disconnected, (ii) when the patient develops an air leak in the pleural cavity, or (iii) when the pressure and flow conditions are generally outside of the proper parameters.

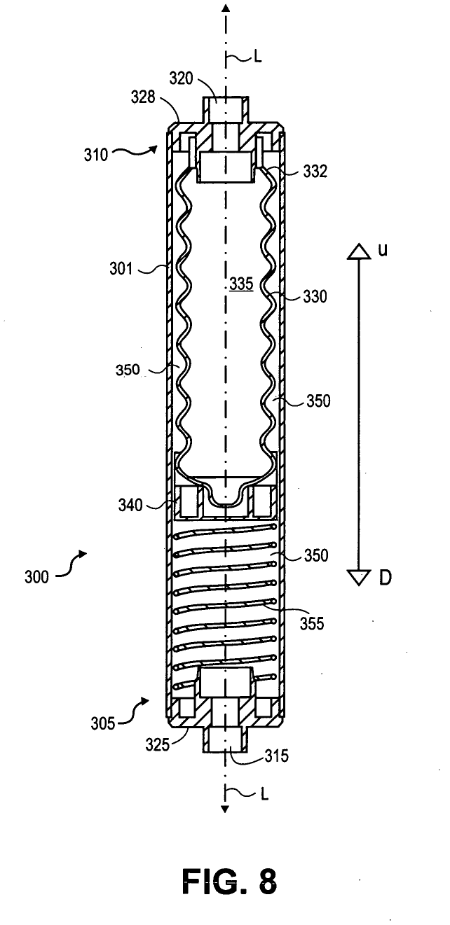

FIG. 8 is a sectional view of another embodiment of a pressure indicator of the present invention. Pressure indicator 300 can be a generally elongate assembly as shown in FIG. 8, having longitudinal axis L. An outer casing 301 has first and second end portions 305 and 310, defining first and second openings 315 and 320, respectively. The outer casing 301 can be cylindrical, or some other shape, such as a rectangular prism, or other suitable elongate three-dimensional volume. The openings 315 and 320 can be disposed on respective end caps 325 and 328, respectively, or could be disposed in the wall of casing 301. In the embodiment shown in FIG. 8, a flexible element 330 is disposed inside the outer casing 301 defining a base open end 332 being attached proximate the second end portion 310 of the outer casing 301 around the second opening 320 to define a collapsible pressure-holding inner space 335 in fluid communication with the second opening 320.

An indicator element in the form of an indicator cap 340 is disposed inside the outer casing configured to translate along the longitudinal axis L, in the directions U and D as shown. The flexible element 330 can be a collapsible and expandable membrane such as a bellows element, being contracted and collapsed along the direction U, and expanded along direction D. The flexible element 330 can thereby retain a particular shape, and hence a particular volume in space 335, for a given longitudinal position along axis L. The indicator element 340 can be attached or coupled to the tip end of the flexible element 330 as shown in FIG. 8, or can be simply pressed against the flexible element by the action of adjacent parts or elements. The flexible element 330, indicator element 340 and outer casing 301 together define a variable interior space 350 inside the outer casing 301 in fluid communication with the first opening 315.

A force resistance element 355 is disposed inside the outer casing 301. This force resistance element can be linear or non-linear, and have a variety of structures which provide a reactive force to the displacement of the indicator cap 340. In one embodiment of the present invention, force resistance element 355 can be a compressive spring, disposed between the indicator cap 340 and end cap 325 at the first end portion 305 of outer casing 301. The expansion of the compressive spring 355 will cause the flexible bellows 330 to contract along direction U. When the first opening 315 is coupled to a first pressure, and the second opening 320 is coupled to a second pressure, these will be transmitted to the pressure holding spaces 335 and 350. If the first pressure (in space 350) is less than the second pressure (in space 335), then the flexible bellows 330 will expand and will cause indicator cap 340 to translate along direction D, which will be resisted by the reactive force of compression spring 355. As the pressure differential between spaces 350 and 335 increases, the bellows 330 will increase in volume, causing the indicator cap 340 to move in direction D ever more, which movement will be brought to an equilibrium by a relatively higher restoring force in direction U by the compression spring 355.

In a chest drainage unit of the present invention, such as shown in FIG. 7, indicator assembly 300 can be substituted for indicator assembly 100, where opening 315 in assembly 300 could be coupled with a conduit such as communication tubing 109 to be in direct fluid communication with a collection chamber 40 for direct reading of patient pressure. Opening 320 in indicator assembly 300 could be coupled to ambient room air, which, when suction is applied to the chest drainage device, will cause relatively low, or negative, pressure to build in space 350 inside indicator assembly 300, reflecting the pressure conditions at the patient end. Ambient room air pressure, being relatively high, or positive, will be present in space 335 inside the bellows 330. This will create a pressure differential which will urge the bellows element 330 to expand and move the indicator cap along direction D, which, as explained above, could be resisted with a compression spring force resistance element 355 which is calibrated a priori to resist a given displacement of indicator cap 340 as a function of the pressure differential—or the pressure inside space 350 directly communicating with the pressure inside the patient. The assembly 300 can thereby function as a true direct pressure gauge for a chest drainage unit. The coupling of either opening 315 or 320 can be made with a connection that runs either inside or outside of the structure of the overall chest drainage assembly.

Alternative embodiments and arrangements of the flexible bellows element 330 and force resistance element 355 are possible. The key principle of the present invention is that the force resistance element 355 is capable of resisting the translation of the indicator element along at least one direction along the longitudinal axis, either direction U or D shown in FIG. 8, when a pressure differential exists between the first and second openings 315 and 320, and hence between spaces 335 and 350. The force resistance element 355 could be a spring in tension, such that, prior to the application of any pressure differential, such a tension spring 355 would cause the flexible bellows 330 to be fully expanded and indicator cap 340 would be furthest along direction D. This time, if a lower pressure were applied to second opening 320 and transmitted into space 335 inside bellows 330, and a higher pressure were applied to first opening 315 and transmitted into space 355, the bellows 330 would contract, indicator cap 340 would translate in direction U, and the movement thereof could be resisted by the restoring tension force of tension spring 355. Again, all displacements and restoring forces could be calibrated as a function of the pressure differential. Similar to the previous embodiments discussed above, a printed surface element wrapped around an exterior surface of the outer casing 301 could be added, said printed surface element having markings to indicate pressure. In addition, a light-absorbing or glowing material could be applied to the elements of the indicator assembly 300, such as the indicator cap 340, or an inside surface the outer casing 301, to allow a viewer to easily ascertain the position of the indicator cap 340, and hence a pressure reading, in low light environments.

An alternative embodiment could also exist, making a slight change in force resistance element 125 shown in the embodiment of FIG. 6A. The force resistance element 125 could be a tension spring instead of a compression spring as discussed above. This would cause bellows 130 to be in a fully contracted or collapsed state prior to the application of any pressure differential between openings 108 and 112. If opening 112 were coupled via a conduit to the collection chamber or patient pressure holding space in the chest drainage unit, transmitting a low, or negative pressure, and opening 108 were exposed to a higher pressure, the bellows 130 would expand, indicator cap 140 would translate along axis L and could be resisted by a restoring force exerted by tension spring 125.

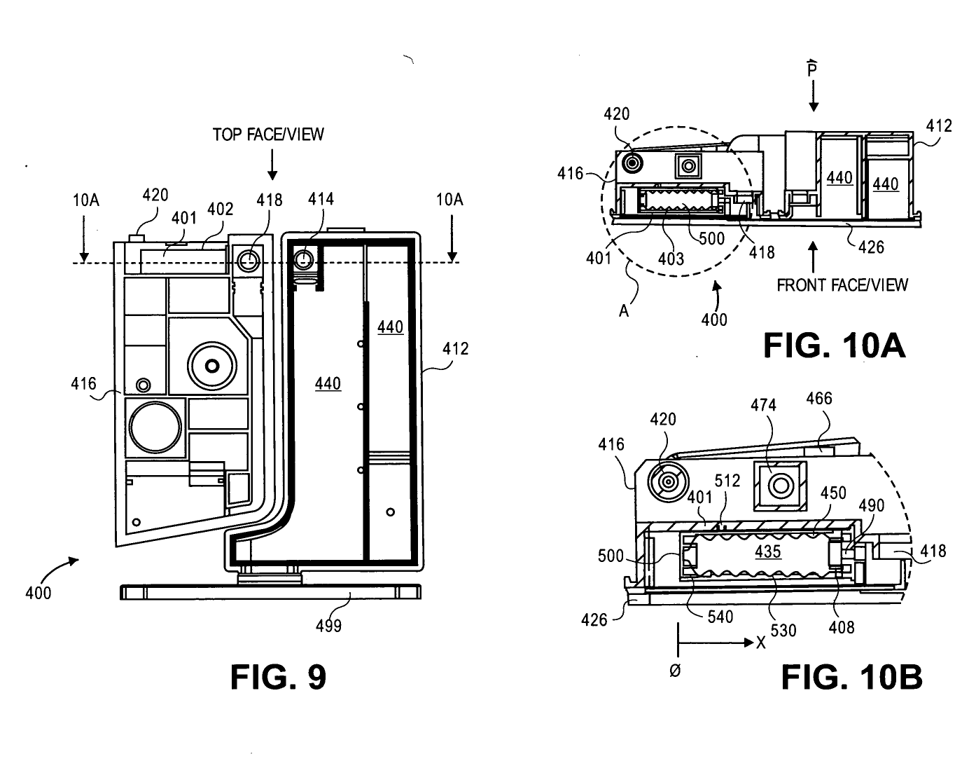

FIG. 9 is a front sectional view of another embodiment of a chest drainage assembly in accordance with the principles of the present invention. In FIG. 9, the face plate 26 shown in FIG. 1 is not shown for ease of viewing and understanding the overall structure of the invention. Similar to the embodiments shown in FIGS. 1 and 2, the chest drainage assembly 400 shown in FIG. 9 is a modular assembly and includes a collection module 412, and a flow control module 416. Collection module 412 operates nearly the same way as collection module 12 shown in FIGS. 1-2, and defines a collection chamber 440, and has an inlet port for receiving fluid suctioned from a patient, and an exit port 414 whereby fluid in the collection chamber 440 can exit the collection module 412. Flow control module 416 in turn functions very similarly to flow control module 16 shown in FIGS. 1-2, and defines an entry port 418 for receiving the suction flow from the collection chamber 440 in collection module 412, and has a suction port 420 for coupling the overall assembly 400 to a suction source. A flow coupling (not shown) is provided between the exit port 414 and the entry port 418. A pressure regulation module similar to that of pressure regulation module 24 can be sealingly coupled to the flow control module 416 and can be positioned in an enclosure defined by the walls and housing geometry of the flow control module 416, similar to the configuration as shown in FIG. 1 for flow control module 16 with pressure regulation module 24. A face plate is placed on the frontal face of the collection module 412 and flow control module 416, the frontal face being defined substantially parallel to the plane of the section view in FIG. 9, looking out from the page. A stand 499 is further provided such that the overall assembly 400 can rest on said stand 499 to remain upright against the direction of gravity g.

One of the principal differences between the embodiments shown in FIGS. 1-4, and that of FIG. 9, is that the pressure indicator device assembly or gauge similar to pressure indicator 100 shown in FIGS. 5-6 is disposed inside a space 401 defined by flow control module 416 having a first window 402 as shown in FIG. 9. A pressure indicator 500 (not shown in FIG. 9) can be positioned in a horizontal orientation, perpendicular to the gravity vector \vec{g}, within space 401. The window 402 allows for a viewer to view the pressure indicator gauge from the frontal face or view, wherein a face plate covering the flow control module 416 includes a corresponding window element allowing for a direct viewing of window 402. This provides a first window and viewing angle for viewing a pressure indicator element such as pressure gauge assembly 100 in a chest drainage assembly 400.

FIG. 10A is a top view of the chest drainage assembly shown in FIG. 9, taken along section 10A-10A as indicated in FIG. 9. By “top view” it is meant herein to mean a view parallel to the plane of the sectional view shown in FIG. 1A, said plane including vector \vec{P} and being substantially perpendicular to gravity vector \vec{g}. This top view shown in FIG. 10A is therefore a cut-away view of the “TOP FACE” of the assembly 400, the “FRONT FACE” being the view normal to and directly facing the face plate 426, the direction of the front face view being parallel but opposite to vector \vec{P}. The top face or top view is therefore the view facing in the direction of arrows 10A-10A shown in FIG. 9. A flow coupling similar to that of flow coupling 22 in FIGS. 1-2 is omitted from the view shown in FIG. 10A of assembly 400, to allow for better clarity in viewing the invention. Pressure indicator gauge 500 is disposed in space 401 and is also viewable from a second window 403 defined by the flow control module 416, which window is substantially parallel to the top face plane. The plane of the second window 403 is therefore rotated by about 90 degrees from the first window 402, and provides for another display and viewing angle for viewing the pressure indicator gauge 100. Thus, the flow control module 416, being a part of the overall chest drainage assembly housing body enclosing the components of the chest drainage assembly 400, provides for a plurality of windows 402, 403, which provide a plurality of viewing angles whereby the pressure indicator gauge or means 100 can be viewed when in operation.

The pressure indicator gauge 500 has an open end which couples to a space in flow control module 416 directly proximate the entry port 418 where fluid is received by the flow control module 416 from collection module 412 (via a flow coupling which is not shown for clarity). An additional more detailed view of this configuration is shown in FIG. 10B. FIG. 10B is an enlarged view of the pressure gauge display in the top view of the chest drainage assembly shown in FIG. 10A, taken within circle “A” in FIG. 10A. The pressure indicator gauge or device 500 includes a bellows element 530 and indicator element or cap 540, each of which are similar to bellows element 130 and indicator cap 140, respectively, shown in FIGS. 5-6. The pressure indicator 500 includes a base end 408 which places the interior or inner space 435 of bellows 530 in fluid communication with the space proximate entry port 418 via a coupling or conduit or channel 490 defined by the flow control module 416. In this manner, any negative or vacuum pressure inside of collection chamber 440 in collection module 412 is transmitted to the interior of bellows 530 into space 435. A compression spring (not shown) similar to compression spring 125, or another suitable force resistance element, is disposed inside of bellows 530, and resists the translation of the indicator cap 540 in the direction “X” as shown, starting from an equilibrium (zero gauge pressure) position “Ø” as shown in FIG. 10B. When vacuum or negative gauge pressure builds in collection chamber 440 and hence in space 435 in bellows 530, the indicator cap 540 progressively translates in the direction X, due to a pressure differential between the negative gauge pressure inside space 435 and the higher pressure in the rest of space 401, which, when the pressure indicator gauge 500 is placed therein, results in a variable interior space 450 similar to the variable interior space 150 discussed with respect to indicator gauge 100 in FIGS. 6A and 6B. Variable interior space 450 can be at a higher pressure than vacuum due to exposure to ambient air via opening 512 defined by the outer casing of the pressure indicator gauge 500, similar to the first opening 112 discussed with respect to the gauge 100 in FIGS. 5-6. Opening 512 can be disposed through a corresponding opening aligned therewith and defined by the housing of flow control module 416.

Flow control module 416 can further include a high negativity pressure relief valve opening 466, as well as a positive pressure relief valve sub-assembly 474. The pressure indicator device or gauge within assembly 400 can also include a gauge similar to that of pressure indicator gauge 300 shown in FIG. 8, where instead of a compression spring being inside of a bellows, the compression spring can be oriented outside of the bellows and indicator cap, such that at equilibrium pressures (with no negative gauge pressure in the collection module), the bellows starts at a fully compressed position, rather than fully expanded as shown in FIGS. 10A-10B. In such a configuration, the pressure indicator gauge could be flipped 180 degrees from the view shown in FIGS. 10A-10B, with the indicator cap translating again along direction X, the base of the bellows being attached to the opposite end of space 401 than that of end 408 shown in FIGS. 10A-10B, and the interior of the bellows being a pressure holding space in fluid communication with a channel exposed though the pressure gauge housing to ambient air outside of flow control module 416, in lieu of the particular arrangement of opening 512 as shown in FIG. 10B.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A pressure indicator in a chest drainage assembly, comprising:
a pressure indicator device; and
a chest drainage assembly housing body enclosing the pressure indicator device, the housing body defining a plurality of windows through which the pressure indicator device can be viewed.

2. The pressure indicator in a chest drainage assembly of claim 1,
wherein the housing body defines a front face and a top face, the front face being substantially aligned along a first plane and the top face being substantially aligned along a second plane perpendicular to the first plane, the plurality of windows including a first window defined on the front face and a second window defined on the top face.

3. The pressure indicator in a chest drainage assembly of claim 1, the chest drainage assembly further comprising:
a collection chamber having a patient fluid intake port, and
a conduit coupling the pressure indicator device with the collection chamber, the pressure indicator device reading a pressure in the collection chamber.

4. The pressure indicator in a chest drainage assembly of claim 1, the pressure indicator device further comprising:
an outer casing having a longitudinal axis and first and second end portions defining first and second openings, respectively,
a flexible element disposed inside the outer casing defining a base open end being attached to the second end portion of the outer casing around the second opening to define a collapsible pressure-holding inner space in fluid communication with the second opening,
an indicator element disposed inside the outer casing configured to translate along the longitudinal axis, the flexible element, indicator element and outer casing together defining a variable interior space inside the outer casing in fluid communication with the first opening, and
a force resistance element disposed inside the outer casing to resist the translation of the indicator element along at least one direction along the longitudinal axis when a pressure differential exists between the first and second openings.

5. The pressure indicator in a chest drainage assembly of claim 4, the chest drainage assembly further comprising:
a collection chamber having a patient fluid intake port, and
a conduit coupling the second opening of the outer casing of the pressure indicator device with the collection chamber, the pressure indicator device reading a pressure in the collection chamber.

6. The pressure indicator in a chest drainage assembly of claim 4,
wherein the force resistance element is a linear force resistance element.

7. The pressure indicator in a chest drainage assembly of claim 4,
wherein the flexible element is a collapsible and expandable membrane.

8. A pressure indicator in a chest drainage device, comprising:
a pressure indicator means; and
a housing enclosing the pressure indicator means, the housing defining a plurality of viewing angles for viewing a pressure indicator element in said pressure indicator means.

9. The pressure indicator in a chest drainage device of claim 8,
wherein the housing defines a front face and a top face, the front face being substantially aligned along a first plane and the top face being substantially aligned along a second plane perpendicular to the first plane, the plurality of viewing angles including a first viewing angle through the front face and a second viewing angle through the top face.

10. The pressure indicator in a chest drainage device of claim 9,
wherein the chest drainage device defines a top portion and a bottom portion, the bottom portion defining a surface upon which the chest drainage device can stand, the top face being disposed on the top portion of the chest drainage device and substantially opposite the surface upon which the chest drainage device can stand.

11. The pressure indicator in a chest drainage device of claim 8, further comprising:
a collection chamber having a patient fluid intake port, and
a conduit coupling the pressure indicator means with the collection chamber, the pressure indicator means reading a pressure in the collection chamber.

12. A chest drainage assembly, comprising:
a pressure gauge having pressure indicator element translating along a longitudinal axis;
an assembly housing enclosing the pressure gauge, the assembly housing defining a top face defining a display for viewing the pressure indicator element.

13. The chest drainage assembly of claim 12,
wherein the assembly housing body defines a front face, the front face being substantially aligned along a first plane and the top face being substantially aligned along a second plane perpendicular to the first plane, the front face defining an additional display for viewing the pressure indicator element.

14. The chest drainage assembly of claim 12, further comprising:
a collection chamber having a patient fluid intake port, and
a conduit coupling the pressure gauge with the collection chamber, the pressure gauge reading a pressure in the collection chamber.

15. The chest drainage assembly of claim 12, the pressure gauge further comprising:
an outer casing aligned along the longitudinal axis and having first and second end portions defining first and second openings, respectively,
a flexible element disposed inside the outer casing defining a base open end being attached to the second end portion of the outer casing around the second opening to define a collapsible pressure-holding inner space in fluid communication with the second opening,
the pressure indicator element being disposed inside the outer casing, the flexible element, pressure indicator element and outer casing together defining a variable interior space inside the outer casing in fluid communication with the first opening, and
a force resistance element disposed inside the outer casing to resist the translation of the indicator element along at least one direction along the longitudinal axis when a pressure differential exists between the first and second openings.

16. The chest drainage assembly of claim 15, the chest drainage assembly further comprising:
a collection chamber having a patient fluid intake port,
the second opening of the outer casing of the pressure gauge being coupled in fluid communication with the collection chamber, the pressure gauge reading a pressure in the collection chamber.

17. The chest drainage assembly of claim 15,
wherein the force resistance element is a linear force resistance element.

18. The chest drainage assembly of claim 15,
wherein the flexible element is a collapsible and expandable membrane.
